(19) United States
(12) Patent Application Publication
(10) Pub. No.: US 2021/0310010 A1
(43) Pub. Date: Oct. 7, 2021

(54) Plasmid Vector for Expressing mRNA in Vitro, Construction Method and Application Thereof

(71) Applicant: Shenzhen NeoCura Biotechnology Corporation, Shenzhen (CN)

(72) Inventors: Youdong PAN, Shenzhen (CN); Qi SONG, Shenzhen (CN); An XIAO, Shenzhen (CN); Yi WANG, Shenzhen (CN); Ji WAN, Shenzhen (CN); Gang LIU, Shenzhen (CN); Ying WEN, Shenzhen (CN)

(73) Assignee: Shenzhen NeoCura Biotechnology Corporation, Shenzhen (CN)

(21) Appl. No.: 17/011,994

(22) Filed: Sep. 3, 2020

(30) Foreign Application Priority Data
Apr. 3, 2020 (CN) 202010259281.8

(51) Int. Cl. C12N 15/66; C12N 15/65

(52) U.S. Cl. CPC C12N 15/66; C12N 15/65

(57) ABSTRACT

A plasmid vector for expressing mRNA in vitro, a construction method and an application thereof are provided. The plasmid vector includes a poly(adenyl deoxyribonucleotide) (poly(dA)) fragment formed by more than 30 adenyl deoxyribonucleotides at the 3′-end tail of a gene to be inserted for expression. The plasmid vector for expressing mRNA in vitro in the present invention can express a target protein gene and a poly(A) formed by 60 adenyl ribonucleotides, and the expressed mRNA directly possesses the poly(A) without additional tailing operation. In addition, the mRNA transcribed in vitro shows stronger mRNA stability and higher protein expression ability after being transfected into cells.

### CROSS REFERENCE TO THE RELATED APPLICATIONS

This application is based upon and claims priority to Chinese Patent Application No. 202010259281.8, filed on Apr. 3, 2020, the entire contents of which are incorporated herein by reference.

### TECHNICAL FIELD

The present invention relates to the technical fields of molecular biology, cell culture, immunobiology, and image analysis. More specifically, the present invention includes: constructing a plasmid vector pNeoCura-Exp060 capable of expressing mRNA in cells by replacing an original sequence formed by 30 adenyl deoxyribonucleotides in an existing plasmid with a poly(adenyl deoxyribonucleotide) fragment formed by 60 adenyl deoxyribonucleotides via restriction endonuclease digestion and ligation; then, inserting a green fluorescent protein (eGFP) gene fragment into the vector to form an example plasmid, followed by transforming the example plasmid into an appropriate Escherichia coli strain, extracting the plasmid after cultivation, linearizing the plasmid in vitro by restriction enzymes, and carrying out a transcribing to obtain an example mRNA having a poly(adenyl ribonucleotide) tail formed by 60 adenyl ribonucleotides; next, transfecting the example mRNA into cells; and finally, quantitatively evaluating the stability and translation ability of mRNA expressed by this kind of vector by detecting the content of the mRNA and the intensity of the expressed eGFP at different time points.

### BACKGROUND

Messenger ribonucleic acid (mRNA) is an important part of eukaryotic gene expression and plays a crucial role in the central dogma of DNA-(transcription)-mRNA-(translation)-protein. Mature mRNA consists of a 5′-cap, a 5′-untranslated region (5′-UTR), a protein coding sequence (CDS), a 3′-untranslated region (3′-UTR), and a 3′-poly(adenyl ribonucleotide) (3′-poly(A) tail). Among them, the 5′-cap and the 3′-poly(A) tail play important roles in the stability of mRNA in vivo or in cultured cell lines, and then affect the efficiency of mRNA translation into proteins or peptides.

At present, in the field of molecular biology, numerous DNA plasmid vectors are designed to transcribe mRNA in vitro as a template (then, this mRNA can be used in biomedical research, clinical applications and so on; to some extent, the mRNA obtained by artificial transcription in vitro mimics the mRNA transcribed in eukaryotic organisms, where the 5′-cap thereof is usually provided by the in vitro transcription kit, while the 3′-poly(A) tail comes from the poly(adenyl deoxyribonucleotide) sequences (also designated as 3′-poly(dA)) contained in the DNA vectors.

Both the 3′-poly(A) tails of natural mRNA and synthetic mRNA can protect mRNA from degrading by the exonucleases in organisms or cells. Currently, most of the vectors for in vitro transcription used in academia and industry contain a 3′-poly(dA) template merely formed by 30 adenyl deoxyribonucleotides.

### SUMMARY

Generally, for academic research, there is not much demand for the addition of long poly(A) tails while transcribing mRNA in vitro. In most experiments, 30 adenyl ribonucleotides are enough. If a longer poly(A) tail is needed in an experiment, a plasmid template without poly(dA) can be used, and the longer poly(A) tail can be added after transcription using an additional commercial tailing kit. However, for the plasmid vectors used in industrial production, performing the transcription and the tailing separately is unacceptable in some cases.

The objective of the present invention is to construct a vector for in vitro transcription of mRNA having a longer 3′-poly(A) tail template that can improve the stability and translation ability of the resulting mRNA in vivo or in cells. Therefore, in the present invention, a plasmid vector capable of adding a poly(A) tail formed by 60 adenyl ribonucleotides to mRNA during the transcription is constructed. It was proved that the mRNA with a longer 3′-poly(A) tail has better stability and stronger ability to translate into a protein or peptide.

Specifically, based on pNeoCura-Exp060, the present invention is implemented by replacing an original 3′-poly(dA) segment formed by 30 adenyl deoxyribonucleotides with a similar segment formed by 60 adenyl deoxyribonucleotides. In the present invention, the 3′-poly(dA) segment formed by 30 adenyl deoxyribonucleotides in the pSP64-Poly(A) vector is removed by the restriction enzymes SacI and EcoRI, and an artificial sequence including an SacI restriction site sticky end, a KpnI restriction site, an XhoI restriction site, a poly(dA) segment formed by 60 adenyl deoxyribonucleotides, an MluI restriction site, and an EcoRI restriction site sticky end is ligated and inserted to obtain a pNeoCura-Exp060 plasmid vector.

In a first aspect of the present invention, a plasmid vector for expressing mRNA in vitro is provided, including a poly(adenyl deoxyribonucleotide) (poly(dA)) fragment formed by more than 30 adenyl deoxyribonucleotides at the 3′-end tail of a gene to be inserted for expression.

In some embodiments of the present invention, the poly(dA) fragment has 60 adenyl deoxyribonucleotides.

In some embodiments of the present invention, the plasmid vector further includes sequences of the pSP64-Poly(A) vector other than the 3′-poly(dA) segment formed by 30 adenyl deoxyribonucleotides and a sequence between an XhoI restriction site and an XbaI restriction site.

In some embodiments of the present invention, the plasmid vector further includes a promoter sequence.

In some embodiments of the present invention, the promoter sequence is T7.

In some embodiments of the present invention, the plasmid vector further includes a target protein gene.

In some embodiments of the present invention, the target protein gene is a green fluorescent protein gene.

In a second aspect of the present invention, a method for constructing the plasmid vector for expressing mRNA in vitro according to the first aspect, including the following steps:

S1, removing the 3′-poly(dA) segment formed by 30 adenyl deoxyribonucleotides in the pSP64-Poly(A) vector by restriction enzymes SacI and EcoRI, and ligating and inserting an artificial sequence including an SacI restriction site sticky end, a KpnI restriction site, an XhoI restriction site, a poly(dA) segment formed by 60 adenyl deoxyribonucleotides, an MluI restriction site, and an EcoRI restriction site sticky end to obtain a pNeoCura-Exp060 plasmid vector; and

S2, removing a 30-bp fragment in the pNeoCura-Exp060 by restriction enzymes XbaI and XhoI, and then ligating and inserting an artificial sequence including an XbaI restriction site sticky end, a T7 RNA polymerase recognition fragment, an eGFP coding expression fragment, and an XhoI restriction site sticky end to obtain an example plasmid pNeoCura-Exp060-eGFP for transcription expression in vitro.

In some embodiments of the present invention, the following steps are further included:

S11, digesting the pSP64-Poly(A) plasmid by restriction enzymes and recovering a long fragment, including;

mixing the pSP64-Poly(A) plasmid with the restriction enzymes including SacI-HF and EcoRI-HF, CutSmart Buffer, and water in the following ratio:

100 ng of pSP64-Poly(A),

0.5 μL of SacI-HE

0.5 μL of EcoRI-HF,

1 μL of CutSmart Buffer, and

making up to 10 μL with water; and

placing the mixture at 37° C. for 4 h, and then performing an electrophoretic separation on a 1.5% agarose gel, and recovering fragments with a length of about 4000 bp by a DNA gel recovery kit and then dissolving in 20 μL of Tris-HCl buffer.

In some embodiments of the present invention, the following steps are further included:

S12, synthesizing an insertion sequence with a poly(dA) segment formed by 60 adenyl deoxyribonucleotides, including:

dissolving two DNA single-stranded sequences including a DNA sequence 1 and a DNA sequence 2 in Tris-HCl buffer to reach a final concentration of 100 ng/μL, respectively, taking 5 μL each to mix, and then heating to 95° C. by a heating block, followed by naturally cooling to room temperature to obtain the insertion sequence double-stranded DNA fragment including a SacI restriction site sticky end, a KpnI restriction site, an XhoI restriction site, the 3′-poly(dA) segment formed by 60 adenyl deoxyribonucleotides, an MluI restriction site, and an EcoRI restriction site sticky end;

the DNA sequence 1:
5′-CGGTACCCTCGAGAAAAAAAAAAAAAAAAAAAAAAAAAAAAAAAAA
AAAAAAAAAAAAAAAAAAAAAAAAAAAAAAACGCGTG-3′ (as
shown in SEQ ID No: 1);
and
the DNA sequence 2:
5′-AATTCACGCGTTTTTTTTTTTTTTTTTTTTTTTTTTTTTTTTTTTT
TTTTTTTTTTTTTTTTTTTTTTTTTCTCGAGGGTACCGAGCT-3′
(as shown in SEQ ID No: 2).

In some embodiments of the present invention, the following steps are further included:

S13, ligating to obtain the pNeoCura-Exp060 vector and amplifying, including:

mixing the above fragments with T4 ligase and T4 Buffer in a T4 ligase kit in the following ratio:

8 μL of the digested and recovered pSP64-Poly(A) fragment,

0.5 μL of the insertion sequence double-stranded DNA fragment,

0.5 μL of T4 ligase, and

1 μL of T4 Buffer; and

placing the mixture at 16° C. for 1 h.

In some embodiments of the present invention, the following steps are further included:

S21, digesting the pNeoCura-Exp060 plasmid by restriction enzymes and recovering a long fragment, including:

mixing the pNeoCura-Exp060 plasmid with the restriction enzymes including XbaI and XhoI, CutSmart Buffer, and water in the following ratio:

100 ng of pNeoCura-Exp060,

0.5 μL of XbaI,

0.5 μL of XhoI,

1 μL of CutSmart Buffer, and

making up to 10 μL with water; and

placing the mixture at 37° C. for 4 h, and then performing an electrophoretic separation on a 1.5% agarose gel, and recovering fragments with a length of about 4000 bp by the DNA gel recovery kit and then dissolving in 20 μL of Tris-HCl buffer.

In some embodiments of the present invention, the following steps are further included:

S22, preparing a T7-EGFP gene amplified restriction fragment, including:

dissolving DNA single-stranded sequences including a DNA sequence 3 and a DNA sequence 4 in Tris-HCl buffer to reach a final concentration of 10 μmmol/L, respectively;

the DNA sequence 3:
5′-ATCGTCTAGATAATACGACTCACTATAGGGATGGTGAGCAAGGGCG
AGGA-3′ (as shown in SEQ ID No: 3),
and
the DNA sequence 4:
5′-ATCGCTCGAGTTACTTGTACAGCTCGTCCATGC-3′ (as shown
in SEQ ID No: 4);

and

mixing the above fragments with the PCR template pcDNA-EGFP plasmid and Taq MasterMix in the following ratio:

10 ng of the PCR template,

0.5 μL of the DNA sequence 3,

0.5 μL of the DNA sequence 4,

10 μL of Taq MasterMix, and

making up to 20 μL with water; and

after PCR amplification, performing an electrophoretic separation on the reaction products with a 1.5% agarose gel, and recovering fragments with a length of about 780 bp by the DNA gel recovery kit and then dissolving in 20 μL of Tris-HCl buffer;

mixing the above fragments with restriction enzymes including XbaI and XhoI, and CutSmart Buffer in the following ratio:

18 μL of pNeoCura-Exp060,

0.5 μL of XbaI,

0.5 μL of XhoI, and

1 μL of CutSmart Buffer; and

placing the mixture at 37° C. for 4 h, and then performing an electrophoretic separation on a 1.5% agarose gel, and recovering fragments with a length of about 780 bp by the DNA gel recovery kit and then dissolving in 20 μL of Tris-HCl buffer to obtain the T7-EGFP gene amplified restriction fragment.

In some embodiments of the present invention, the following steps are further included:

S23, ligating to obtain the pNeoCura-Exp060-T7-EGFP plasmid and amplifying, including:

mixing the above fragment with T4 ligase and T4 Buffer in the T4 ligase kit in the following ratio:

1 μL of the digested and recovered long pNeoCura-Exp060 fragment,

7.5 μL, of the EGFP gene amplified fragment,

0.5 μL of T4 ligase, and

1 μL of T4 Buffer; and

placing the mixture at 16° C. for 1 h.

In a third aspect of the present invention, an application of the plasmid vector for expressing mRNA in vitro described in the first aspect is provided.

The advantages of the present invention are as follows.

The plasmid vector for expressing mRNA in vitro in the present invention can express a target protein gene and a poly(A) formed by 60 adenyl ribonucleotides, and the expressed mRNA directly possesses the poly(A) without additional tailing operation. In addition, the mRNA transcribed in vitro shows stronger mRNA stability and higher protein expression ability after being transfected into cells.

### BRIEF DESCRIPTION OF THE DRAWINGS

FIG. 1 shows the DNA sequence 1, the DNA sequence 2, and the insertion sequence double-stranded DNA fragment including a SacI restriction site sticky end, a KpnI restriction site, an XhoI restriction site, a 3′-poly(dA) segment formed by 60 adenyl deoxyribonucleotides, an MluI restriction site, and an EcoRI restriction site sticky end, formed by the DNA sequence 1 and the DNA sequence 2 after annealing;

FIG. 2 shows a process of ligating the pSP64-Poly(A) plasmid with the above insertion sequence double-stranded DNA fragment to obtain a pNeoCura-Exp060 vector;

FIG. 3 shows a process of ligating the pNeoCura-Exp060 vector plasmid with a T7-EGFP gene amplified restriction fragment to obtain a pNeoCura-Exp060-T7-EGFP plasmid;

FIG. 4A shows an electrophoretic band comparison of a stability test of mRNA transcribed from the pNeoCura-Exp060-T7-EGFP plasmid and a control plasmid at 0 h, 4 h, and 8 h after transfecting HEK 293T cells;

FIG. 4B shows the results of qPCR detection of remaining mRNA of mRNA transcribed from the pNeoCura-Exp060-T7-EGFP plasmid and a control plasmid at 0 h, 4 h, and 8 h after transfecting HEK 293T cells;

FIG. 4C shows an electrophoretic band comparison of proteins expressed after transfecting HEK 293T cells by mRNA transcribed from the pNeoCura-Exp060-T7-EGFP plasmid and a control plasmid; and

FIG. 4D shows the results of a protein expression ability test (Western blot detection of EGFP) of mRNA transcribed from the pNeoCura-Exp060-T7-EGFP plasmid and a control plasmid at 0 h, 4 h, and 8 h after transfecting HEK 293T cells.

### DETAILED DESCRIPTION OF THE EMBODIMENTS

The present invention is illustrated by the following specific embodiments. Those skilled in the art can easily understand the other advantages and values of the present invention from the contents in the present disclosure. The present invention can also be implemented or applied through other different specific embodiments. The details in the present disclosure can further be modified or changed without deviating from the spirit of the present invention based on different viewpoints and applications.

Before further describing the specific embodiments of the present invention, it should be understood that the protection scope of the present invention is not limited to the following specific embodiments. Also, it should be understood that the terminologies used in the embodiments of the present invention are intended to describe the specific embodiments rather than to limit the protection scope of the present invention.

When an embodiment uses a numerical range, it should be understood that unless otherwise stated in the present invention, two endpoints of each numerical range and any one value between the two endpoints can be selected. Unless otherwise defined, all technical and scientific terms used in the present invention have the same meaning as generally known by those skilled in the art. In addition to the specific methods, devices and materials used in the embodiments, those skilled in the art can also use any method, device and material in the prior art similar to or equivalent to the method, device and material described in the embodiments of the present invention to realize the present invention according to the mastery of the existing techniques and the disclosure of the present invention.

### Embodiment 1

(1) Construction of vector pNeoCura-Exp060 for transcription expression in vitro

To overcome the shortcomings of the existing vector pNeoCura-Exp060 for in vitro transcription that the stability of the transcribed mRNA is not strong enough and the translation ability is not high enough due to its relatively short length, the present invention solves the problem by replacing an original 3′-poly(dA) segment formed by 30 adenyl deoxyribonucleotides in the pSP64-Poly(A) vector with a similar segment formed by 60 adenyl deoxyribonucleotides.

Specifically, in the present invention, the 3′-poly(dA) segment formed by 30 adenyl deoxyribonucleotides in the pSP64-Poly(A) vector is removed by the restriction enzymes SacI and EcoRI, and an artificial sequence including an SacI restriction site sticky end, a KpnI restriction site, an XhoI restriction site, a 3′-poly(dA) segment formed by 60 adenyl deoxyribonucleotides, an MluI restriction site, and an EcoRI restriction site sticky end is ligated and inserted to obtain a pNeoCura-Exp060 plasmid vector.

(2) Construction of the example plasmid pNeoCura-Exp060-T7-EGFP for transcription expression in vitro and a test of characteristics of its transcribed products in vitro

In the present invention, a fragment with a length of 30 bp in the pNeoCura-Exp060 is removed by the restriction enzymes XbaI and XhoI, and then an artificial sequence including an XbaI restriction site sticky end, a T7 RNA polymerase recognition fragment, an eGFP protein coding expression fragment, and an XhoI restriction site sticky end is ligated and inserted to obtain an example plasmid pNeoCura-Exp060-eGFP for transcription expression in vitro.

After being fully cloned in Escherichia coli, the pNeoCura-Exp060-T7-EGFP plasmid is extracted and purified, linearized by restriction enzyme MluI, and transcribed into mRNA carrying an EGFP coding sequence and a 3′-poly(A) tail formed by 60 adenyl ribonucleotides in vitro using a T7 Ultra in vitro transcription kit.

The present invention uses a liposome transfection technique to transfect the above mRNA into the FMK 293T cell line cultured in vitro, and detects the remaining mRNA content and EGFP protein content in the cell line after 4 h and 8 h, so as to determine the stability and translation expression intensity of the mRNA in the cells.

(1) Main reagents and instruments

| Main reagents in the present invention | Supplier |
|---|---|
| SacI-HF restriction enzyme | New England Biolabs |
| EcoRI-HF restriction enzyme | New England Biolabs |
| XbaI restriction enzyme | New England Biolabs |
| XhoI restriction enzyme | New England Biolabs |
| MluI-HF restriction enzyme | New England Biolabs |
| CutSmart restriction enzyme buffer | New England Biolabs |
| Agarose | New England Biolabs |
| Tris-HCl buffer (pH 8.0) | New England Biolabs |
| DNA gel recovery kit | Qiagen |
| DNA plasmid extraction kit | Qiagen |
| Cell RNA extraction kit | Qiagen |
| M-MLV reverse transcription kit | Thermo Fisher Scientific |
| EGFP real-time fluorescence quantitative | Thermo Fisher Scientific |
| PCR primer-probe set | |
| Cell protein extraction kit | Thermo Fisher Scientific |
| Western blot detection kit | Thermo Fisher Scientific |
| T4 DNA ligation kit | New England Biolabs |
| TOP 10 E. coli chemical competent cell kit | New England Biolabs |
| Taq MasterMix | Thermo Fisher Scientific |
| LB liquid medium pre-made powder | Thermo Fisher Scientific |
| LB-agar solid medium pre-made powder | Thermo Fisher Scientific |
| mMessager mMachine T7 Ultra in vitro | Thermo Fisher Scientific |
| transcription kit | |
| RNA recovery and purification kit | Qiagen |
| HEK 293T cell line | ATCC |
| DMEM cell culture medium | Thermo Fisher Scientific |
| Fetal Bovine Serum (FAB) | Gemini Bio-Products |
| Penicillin-streptomycin mixed solution | Thermo Fisher Scientific |
| (Penicillin/Streptomycin) | |
| PBS buffer | Thermo Fisher Scientific |
| Trypsin/EDTA mixed solution | Thermo Fisher Scientific |
| Lipofectamine 2000 transfection kit | Thermo Fisher Scientific |
| pSP64-Poly(A) plasmid | Addgene |
| pcDNA3-EGFP plasmid | Addgene |
| Main instruments in the present invention | Supplier |
| PCR amplifier | BioRad |
| Gel electrophoresis apparatus | New England Biolabs |
| 16° C. incubator | New England Biolabs |
| 37° C. cell incubator | New England Biolabs |
| −20° C. refrigerator | Zhongke Meiling |
| 4° C. refrigerator | Zhongke Meiling |
| 6-well cell culture plate | Thermo Fisher Scientific |

(2) Experimental methods.

1. Construction of vector pNeoCura-Exp060 for transcription expression in vitro

1.1 Digestion of pSP64-Poly(A) plasmid by restriction enzymes and recovery of long fragments

First, the pSP64-Poly(A) plasmid is mixed with the restriction enzymes including SacI-HF and EcoRI-HF, CutSmart Buffer, and water in the following ratio:

100 ng of pSP64-Poly(A),

0.5 μL of SacI-HF,

0.5 μL of EcoRI-HF,

1 μL of CutSmart Buffer, and

making up to 10 μL with water.

The mixture is mixed at 37° C. for 4 h. Then an electrophoretic separation is performed on a 1.5% agarose gel, and fragments with a length of about 4000 bp are recovered by the DNA gel recovery kit and then dissolved in 20 μL of Tris-HCl buffer.

1.2 Synthesis of an insertion sequence containing a poly(dA) segment formed by 60 adenyl deoxyribonucleotides

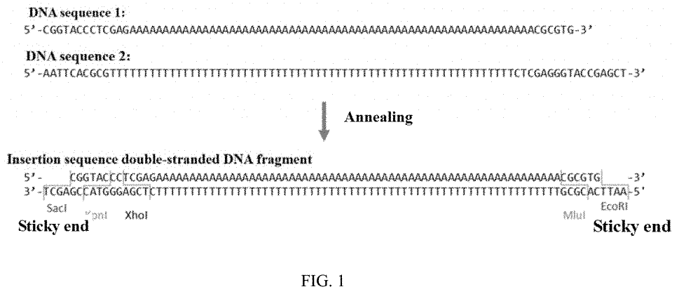

Two DNA single-strand sequences (including DNA sequence 1 and DNA sequence 2) are synthesized by third-party suppliers and dissolved in Tris-HCl buffer to achieve a final concentration of 100 ng/μL, respectively. 5 μL of each solution is taken out and mixed together, then heated to 95° C. with a heating block, and cooled naturally to room temperature, so as to obtain the insertion sequence double-stranded DNA fragment including a SacI restriction site sticky end, a KpnI restriction site, an XhoI restriction site, the 3′-poly(dA) segment formed by 60 adenyl deoxyribonucleotides, an MluI restriction site, and an EcoRI restriction site sticky end (see FIG. 1).

The DNA sequence 1:
5′-CGGTACCCTCGAGAAAAAAAAAAAAAAAAAAAAAAAAAAAAAAAAA
AAAAAAAAAAAAAAAAAAAAAAAAAAAACGCGTG-3′ (as shown
in SEQ ID No: 1),
and
The DNA sequence 2:
5′-AATTCACGCGTTTTTTTTTTTTTTTTTTTTTTTTTTTTTTTTTTTTT
TTTTTTTTTTTTTTTTTTTTTTTTCTCGAGGGTACCGAGCT-3′ (as
shown in SEQ ID No: 2).

1.3 Ligation into pNeoCura-Exp060 vector, amplification and verification

The above fragments are mixed with T4 ligase and T4 Buffer in the T4 ligase kit in the following ratio:

8 μL of the digested and recovered pSP64-Poly(A) fragment,

0.5 μL of the insertion sequence double-stranded DNA fragment,

0.5 μL of T4 ligase, and

1 μL of T4 Buffer.

The mixture is placed at 16° C. for 1 h. The ligated plasmid is then transformed into TOP10 E. coli competent cells according to the standard instructions of third-party suppliers. A predetermined amount of transformed cells are smeared on the LB solid medium plate and cultured at 37° C. for 14-16 h.

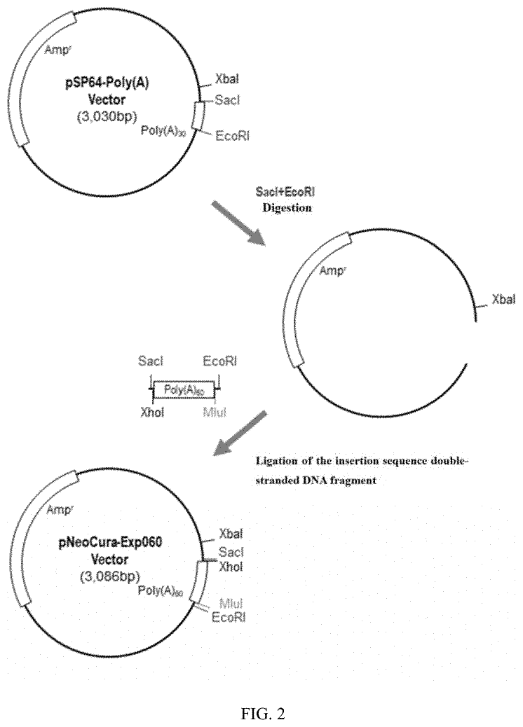

Eight single colonies are selected and placed in 5 mL of LB liquid medium and cultured at 37° C. for 14-16 h. Then, the plasmids are extracted by the DNA plasmid extraction kit and sent to third-party sequencing companies for sequencing with SP6 primers, and the plasmids with the correct sequence are preserved. Thus, the pNeoCura-Exp060 vector is obtained (see FIG. 2).

2. Application example of the pNeoCura-Exp060 vector: construction of example plasmid pNeoCura-Exp060-T7-EGFP for transcription expression in vitro and the test of characteristics of its transcribed products in vitro

2.1 Digestion of the pNeoCura-Exp060 plasmid by restriction enzymes and recovery of long fragments

First, the pNeoCura-Exp060 plasmid is mixed with the restriction enzymes including XbaI and XhoI, CutSmart Buffer, and water in the following ratio:

100 ng of pNeoCura-Exp060,

0.5 μL of XbaI,

0.5 μL of XhoI,

1 μL of CutSmart Buffer, and

making up to 10 μL with water.

The mixture is placed at 37° C. for 4 h. Then, an electrophoretic separation is performed on a 1.5% agarose gel. The fragments with a length of about 4000 bp is recovered by the DNA gel recovery kit and then dissolved in 20 μL of Tris-HCl buffer.

2.2 Preparation of a T7-EGFP gene amplified restriction fragment

DNA single-stranded sequence 3 and sequence 4 are synthesized through third-party suppliers. Among them, the sequence 3 includes 4 protection bases, an XbaI restriction site, a T7 promoter, and the first 20 bases of the EGFP protein coding sequence. The sequence 4 includes 4 protection bases, an XbaI restriction site, an antisense complementary sequence (TTA) of stop codon TAA, and an antisense complementary sequence of the last 20 bases of the EGFP protein coding sequence. The sequence 3 and sequence 4 are dissolved in Tris-HCl buffer to achieve a final concentration of 10 μmmol/L, respectively.

The sequence 3:
5′-ATCGTCTAGATAATACGACTCACTATAGGGATGGTGAGCAAGGGCG
AGGA-3′ (as shown in SEQ ID No: 3).
The sequence 4:
5′-ATCGCTCGAGTTACTTGTACAGCTCGTCCATGC-3′ (as shown
in SEQ ID No: 4).

The above fragments are mixed with PCR template (pcDNA-EGFP plasmid) and Taq MasterMix in the following ratio:

10 ng of the PCR template,

0.5 μL of the DNA sequence 3,

0.5 μL of the DNA sequence 4,

10 μL of Taq MasterMix, and

making up to 20 μL with water.

The following procedure is used to react on the PCR amplifier: 95° C. for 3 min, 30 cycles (95° C. for 20 s, 55° C. for 20 s, and 72° C. for 1 min), and 72° C. for 6 min.

Then, the reaction products are subjected to an electrophoretic separation on a 1.5% agarose gel. The fragments with a length of about 780 bp are recovered by the DNA gel recovery kit and then dissolved in 20 μL of Tris-HCl buffer.

The above fragments are mixed with restriction enzymes including XbaI and XhoI, and CutSmart Buffer in the following ratio:

18 μL of pNeoCura-Exp060,

0.5 μL of XbaI,

0.5 μL of XhoI, and

1 μL of CutSmart Buffer.

The mixture is placed at 37° C. for 4 h. Then, an electrophoretic separation is performed with a 1.5% agarose gel. The fragments with a length of about 780 bp are recovered by the DNA gel recovery kit and then dissolved in 20 μL of Tris-HCl buffer to obtain the T7-EGFP gene amplified restriction fragment.

2.3 Ligation into pNeoCura-Exp060-T7-EGFP plasmid, amplification and verification

The above fragments are mixed with T4 ligase and T4 buffer in the T4 ligase kit in the following ratio:

1 μL of the digested and recovered long pNeoCura-Exp060 fragment,

7.5 μL of the EGFP gene amplified fragment,

0.5 μL of T4 ligase, and

1 μL of T4 buffer.

The mixture is placed at 16° C. for 1 h. The ligated plasmid is then transformed into TOP10 E. coli competent cells according to the standard instructions of third-party suppliers. A predetermined amount of transformed cells are smeared on the LB solid medium plate and cultured at 37° C. for 14-16 h.

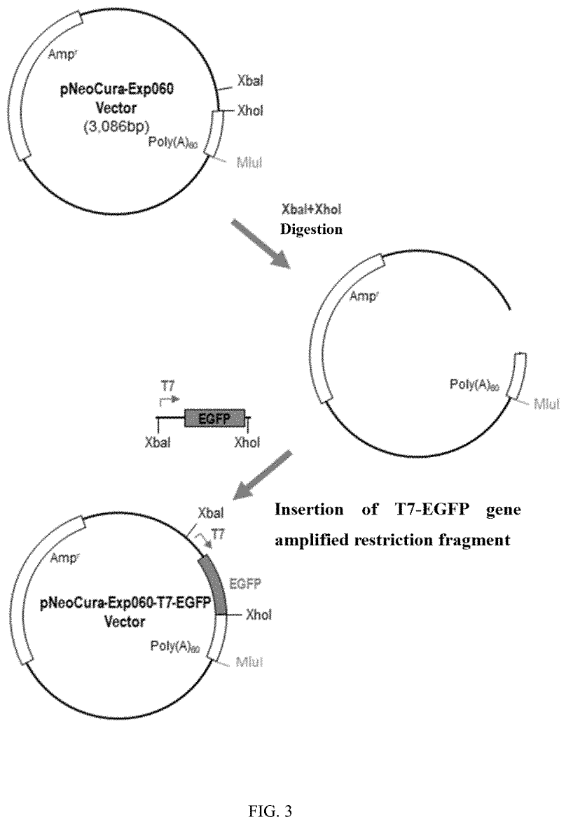

Eight single colonies are selected and placed in 5 mL of LB liquid medium and cultured at 37° C. for 14-16 h. Then, the plasmids are extracted using the DNA plasmid extraction kit and sent to third-party sequencing companies for sequencing with SP6 primers, and the plasmids with the correct sequence are preserved. Thus, the pNeoCura-Exp060-T7-EGFP vector is obtained (see FIG. 3).

Meanwhile, a third-party reference plasmid inserted with a coding EGFP protein coding sequence and a poly(dA) formed by 30 adenyl deoxyribonucleotides is obtained by a similar method as a control (control plasmid).

2.4 Transcriptional synthesis of mRNA encoding EGFP protein in vitro

The pNeoCura-Exp060-T7-EGFP plasmid is mixed with restriction enzyme MluI-HF and CutSmart Buffer in the following ratio:

500 ng of the pNeoCura-Exp060-T7-EGFP plasmid,

0.5 μL, of MluI-HF,

1 μL of CutSmart Buffer, and

making up to 10 μL with water.

The mixture is placed at 37° C. for 4 h, and then is purified and recovered by the DNA gel recovery kit, and dissolved in 20 μL of Tris-HCl buffer to obtain a linearized product of the pNeoCura-Exp060-T7-EGFP plasmid.

The linearized product of the pNeoCura-Exp060-T7-EGFP plasmid is transcribed in vitro and purified by the mMessager mMachine T7 Ultra in vitro transcription kit according to the standard operation provided by the third party, so as to obtain the mRNA (EGFP-poly(A)60 mRNA) encoding the EGFP protein and carrying a poly(A) tail formed by 60 adenyl ribonucleotides.

Meanwhile, using similar methods, the control plasmid is transcribed to obtain mRNA encoding the EGFP protein and carrying a poly(A) tail formed by 30 adenyl ribonucleotides as a control (control mRNA).

2.5. Cell transfection of the mRNA transcribed in vitro and a test of the characteristics thereof

The FMK 293T cell line is inoculated into the 6-well cell culture plate at a density of 5×10⁵ cells per well. 2 mL of complete medium (including DMEM cell culture medium, the fetal bovine serum, and the penicillin-streptomycin mixed solution) is added to each well and cultured overnight at 37° C.

The cells are washed with PBS buffer, suspended with trypsin/EDTA mixed solution, and transfected with 500 ng of the EGFP-poly(A)60 mRNA or the control mRNA by the Lipofectamine 2000 transfection kit according to the standard procedures provided by the third party. After transfection, the cells are cultured at 37° C.

At 4 h and 8 h after transfection, equal amounts of cells are taken, respectively. The total RNA is extracted by the cell RNA extraction kit, and reversed into cDNA by the M-MLU reverse transcription kit. Then, the EGFP real-time fluorescent quantitative PCR primer-probe set is used to detect the remaining amount of the EGFP-poly(A)60 mRNA or the control mRNA by qPCR.

At 4 h and 8 h after transfection, equal amounts of cells are taken, respectively. The total protein is extracted by the cell protein extraction kit. The Western blot detection kit is used to detect the amount of EGFP protein expressed by the EGFP-poly(A)60 mRNA or the control mRNA according to the standard procedures provided by the third party.

(3) Experimental Results

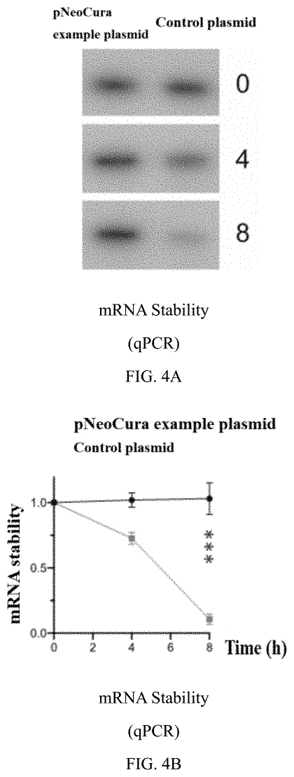

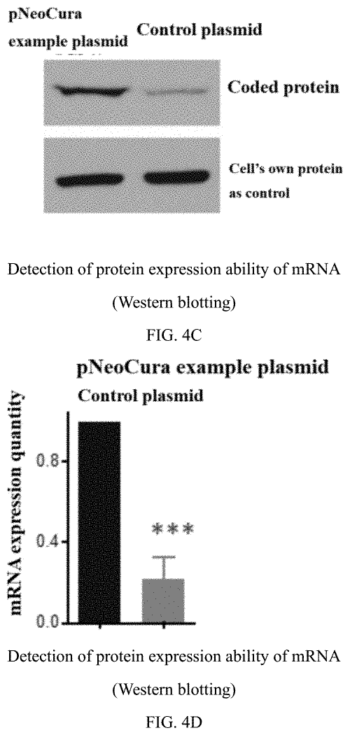

Compared with the control plasmid, the in-vitro transcribed mRNA of the pNeoCura-Exp060-T7-EGFP example plasmid showed stronger mRNA stability (having higher remaining amount of mRNA detected by qPCR) and higher protein expression ability (having stronger EGFP signal detected by Western blot) after transfection into FMK 293T cells (see FIG. 4A-FIG. 4D, where the pNeoCura example plasmid is the pNeoCura-Exp060-T7-EGFP example plasmid).

The preferred specific implementation methods and embodiments of the present invention are described in detail above, but is not used to limit the present invention.

Within the scope of knowledge possessed by those skilled in the art, various changes can be further made without departing from the conception of the present invention.

What is claimed is:

1. A plasmid vector for expressing mRNA in vitro, comprising a poly(adenyl deoxyribonucleotide) (poly(dA)) fragment, wherein the poly(dA) fragment is formed by more than 30 adenyl deoxyribonucleotides at a 3′-end tail of a gene to be inserted for expression.

2. The plasmid vector according to claim 1, wherein the poly(dA) fragment has 60 adenyl deoxyribonucleotides.

3. The plasmid vector according to claim 1, further comprising sequences of a pSP64-Poly(A) vector other than the poly(dA) fragment and a sequence between an XhoI restriction site and an XbaI restriction site.

4. The plasmid vector according to claim 1, further comprising a promoter sequence.

5. The plasmid vector according to claim 4, wherein the promoter sequence is T7.

6. The plasmid vector according to claim 1, further comprising a target protein gene.

7. The plasmid vector according to claim 6, wherein the target protein gene is a green fluorescent protein gene.

8. A method for constructing the plasmid vector for expressing mRNA in vitro according to claim 1, comprising the following steps:
S1, removing the poly(dA) fragment in a pSP64-Poly(A) vector by restriction enzymes SacI and EcoRI, and ligating and inserting an artificial sequence including an SacI restriction site sticky end, a KpnI restriction site, an XhoI restriction site, a 3′-poly(dA) segment formed by 60 adenyl deoxyribonucleotides, an MluI restriction site, and an EcoRI restriction site sticky end to obtain a pNeoCura-Exp060 plasmid vector; and
S2, removing a 30-bp fragment in the pNeoCura-Exp060 plasmid vector by restriction enzymes XbaI and XhoI, and then ligating and inserting an artificial sequence including an XbaI restriction site sticky end, a T7 RNA polymerase recognition fragment, an eGFP protein coding expression fragment, and an XhoI restriction site sticky end to obtain an example plasmid pNeoCura-Exp060-eGFP for transcription expression in vitro.

9. The method according to claim 8, further comprising the following steps:
S11, digesting the pSP64-Poly(A) vector by the restriction enzymes SacI and EcoRI and recovering a long fragment, comprising:
mixing the pSP64-Poly(A) vector with restriction enzymes including SacI-HF and EcoRI-HF, CutSmart Buffer, and water in the following ratio to obtain a first mixture:
100 ng of the pSP64-Poly(A),
0.5 μL of the SacI-HF,
0.5 μL of the EcoRI-HF,
1 μL of the CutSmart Buffer, and
making up to 10 μL with water; and
placing the first mixture at 37° C. for 4 h, and then performing an electrophoretic separation on a 1.5% agarose gel, and recovering first fragments with a length of about 4000 bp by a DNA gel recovery kit and then dissolving the first fragments in 20 μL of Tris-HCl buffer; and/or,
S12, synthesizing an insertion sequence with a poly(dA) segment formed by 60 adenyl deoxyribonucleotides, comprising:
dissolving a first DNA single-stranded sequence and a second DNA single-stranded sequence in Tris-HCl buffer to reach a final concentration of 100 ng/μL, respectively, taking 5 μL of the first DNA single-stranded sequence and 5 μL of the second DNA single-stranded sequence to mix to obtain a mixed sequences, and then heating the mixed sequences to 95° C. by a heating block, followed by naturally cooling the mixed sequences to room temperature to obtain an insertion sequence double-stranded DNA fragment including a SacI restriction site sticky end, a KpnI restriction site, an XhoI restriction site, the 3′-poly(dA) segment formed by 60 adenyl deoxyribonucleotides, an MluI restriction site, and an EcoRI restriction site sticky end;
wherein the first DNA single-stranded sequence is as shown in SEQ ID No: 1; and
the second DNA single-stranded sequence is as shown in SEQ ID No: 2; and/or
S13, ligating to obtain the pNeoCura-Exp060 plasmid vector and amplifying, comprising:
mixing the first fragments with T4 ligase and T4 Buffer in a T4 ligase kit in the following ratio to obtain a second mixture:
8 μL of the first fragments,
0.5 μL of the insertion sequence double-stranded DNA fragment,
0.5 μL of the T4 ligase, and
1 μL of the T4 Buffer; and
placing the second mixture at 16° C. for 1 h; and/or
S21, digesting the pNeoCura-Exp060 plasmid vector by the restriction enzymes XbaI and XhoI and recovering a long fragment, comprising:
mixing the pNeoCura-Exp060 plasmid vector with the restriction enzymes including XbaI and XhoI, CutSmart Buffer, and water in the following ratio to obtain a third mixture:
100 ng of the pNeoCura-Exp060 plasmid vector,
0.5 μL of the XbaI,
0.5 μL of the XhoI,
1 μL of the CutSmart Buffer, and
making up to 10 μL with water; and
placing the third mixture at 37° C. for 4 h, and then performing an electrophoretic separation on a 1.5% agarose gel, and recovering second fragments with a length of about 4000 bp by the DNA gel recovery kit and then dissolving the second fragments in 20 μL of Tris-HCl buffer; and/or
S22, preparing a T7-EGFP gene amplified restriction fragment, comprising:
dissolving a third DNA single-stranded sequence and a fourth DNA single-stranded sequence in Tris-HCl buffer to reach a final concentration of 10 μmmol/L, respectively;
wherein the third DNA single-stranded sequence is as shown in SEQ ID No: 3, and
the fourth DNA single-stranded sequence is as shown in SEQ ID No: 4; and
mixing the second fragments with a PCR template pcDNA-EGFP plasmid and Taq MasterMix in the following ratio:
10 ng of the PCR template pcDNA-EGFP plasmid,
0.5 μL of the third DNA single-stranded sequence,
0.5 μL of the fourth DNA single-stranded sequence,
10 μL of the Taq MasterMix, and
making up to 20 μL with water; and
after a PCR amplification, performing an electrophoretic separation on reaction products with a 1.5% agarose gel, and recovering third fragments with a length of about 780 bp by the DNA gel recovery kit and then dissolving the third fragments in 20 μL of Tris-HCl buffer;
mixing the third fragments with restriction enzymes including XbaI and XhoI, and CutSmart Buffer in the following ratio to obtain a fourth mixture:
18 μL of pNeoCura-Exp060,
0.5 μL of the XbaI,
0.5 μL of the XhoI, and
1 μL of the CutSmart Buffer; and
placing the fourth mixture at 37° C. for 4 h, and then performing an electrophoretic separation on a 1.5% agarose gel, and recovering fourth fragments with a length of about 780 bp by the DNA gel recovery kit and then dissolving the fourth fragments in 20 μL of Tris-HCl buffer to obtain the T7-EGFP gene amplified restriction fragment; and/or
S23, ligating to obtain a pNeoCura-Exp060-T7-EGFP plasmid and amplifying, comprising:
mixing the fourth fragments with T4 ligase and T4 Buffer in the T4 ligase kit in the following ratio to obtain a fifth mixture:
1 μL of the fourth fragments,
7.5 μL of a EGFP gene amplified fragment,
0.5 μL of the T4 ligase, and
1 μL of the T4 Buffer; and
placing the fifth mixture at 16° C. for 1 h.

10. A method of use, comprising applying the plasmid vector for expressing mRNA in vitro according to claim 1.

11. The plasmid vector according to claim 2, further comprising sequences of the pSP64-Poly(A) vector other than the poly(dA) fragment and a sequence between an XhoI restriction site and an XbaI restriction site.

12. The plasmid vector according to claim 2, further comprising a promoter sequence.

13. The plasmid vector according to claim 3, further comprising a promoter sequence.

14. The plasmid vector according to claim 2, further comprising a target protein gene.

15. The plasmid vector according to claim 3, further comprising a target protein gene.

16. The plasmid vector according to claim 4, further comprising a target protein gene.

17. The plasmid vector according to claim 5, further comprising a target protein gene.

18. The method according to claim 8, wherein the poly(dA) fragment has 60 adenyl deoxyribonucleotides.

19. The method according to claim 8, wherein the plasmid vector further comprises sequences of a pSP64-Poly(A) vector other than the poly(dA) fragment and a sequence between an XhoI restriction site and an XbaI restriction site.

20. The method according to claim 8, wherein the plasmid vector further comprises a promoter sequence.


SEQUENCE LISTING (4 sequences)

SEQ ID NO: 1 - DNA, 80 bp, Artificial Sequence
  The sequence is synthetized.
cggtaccctcgagaaaaaaaaaaaaaaaaaaaaaaaaaaaaaaaaaaaaaaaaaaaaaaaaaaaaaaaaaaaaacgcgtg

SEQ ID NO: 2 - DNA, 88 bp, Artificial Sequence
  The sequence is synthetized.
aattcacgcgtttttttttttttttttttttttttttttttttttttttttttttttttttttttttttttctcgagggtaccgagct

SEQ ID NO: 3 - DNA, 50 bp, Artificial Sequence
  The sequence is synthetized.
atcgtctagataatacgactcactatagggatggtgagcaagggcgagga

SEQ ID NO: 4 - DNA, 33 bp, Artificial Sequence
  The sequence is synthetized.
atcgctcgagttacttgtacagctcgtccatgc